(19) United States
(12) Patent Application Publication
(10) Pub. No.: US 2009/0043314 A1
(43) Pub. Date: Feb. 12, 2009

(54) ACCESS DEVICE FOR BODY TISSUE AND METHOD OF USING SAME

(75) Inventors: Lars G. Sevensson, Gates Mills, OH (US); Bruce Lytle, Chagrin Falls, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 12/173,688

(22) Filed: Jul. 15, 2008

Related U.S. Application Data
(60) Provisional application No. 60/959,712, filed on Jul. 16, 2007.

(51) Int. Cl. A61B 17/00

(52) U.S. Cl. 606/108

(57) ABSTRACT

A body tissue has oppositely disposed first and second tissue sides with an aperture extending therebetween. An apparatus for selectively permitting access through the body tissue includes a proximal balloon adapted for placement adjacent the first tissue side. A distal balloon is adapted for placement adjacent the second tissue side. An expandable support member is adapted to extend through the aperture and defines a support member lumen providing fluid communication between the first and second tissue sides. The expandable support member is attached to the proximal and distal balloons and has at least one of an inner support member lining and an outer support member cover. A support member valve is located within the support member lumen and selectively permits access through the aperture between the first and second tissue sides. A closure device is provided for permanently occluding the support member lumen.

### RELATED APPLICATION

This application claims the benefit of U.S. Provisional Patent Application Ser. No. 60/959,712, filed Jul. 16, 2007 which is incorporated herein by reference.

### TECHNICAL FIELD

The present invention relates to an apparatus and method for use of an access device for body tissue and, more particularly, to an apparatus and method for selectively permitting access through a body tissue.

### BACKGROUND OF THE INVENTION

With the growing availability of technologically advanced surgical tools, minimally invasive surgeries are becoming common. One example of a minimally invasive type of surgery is a percutaneous (“through the skin”) procedure.

Percutaneous surgery involves piercing of the patient's skin at an appropriate access point and inserting a tubular port member through the perforation. Various instruments may be extended through the port member to perform surgical operations inside the patient's body, such as gynecological, gastroenterological, or cardiac procedures.

Percutaneous surgery provides several advantages over conventional incision-based surgery. The percutaneous perforations are substantially smaller than the incisions made during conventional operations and are therefore less traumatic to the patient, which leads to an accelerated recovery and convalescence. Hospital stays may be minimized by the generally short recovery period characteristic of percutaneous surgery. As a result, percutaneous surgery is normally less time-consuming and less expensive than analogous conventional surgery.

Percutaneous surgery frequently requires temporary access through one or more body tissues. For example, in a cardiac procedure, a surgeon may need to access a chamber of the heart from an adjacent location within the chest, but without allowing the blood within that heart chamber to escape during or after the surgery. Therefore, the percutaneous equipment and/or techniques must allow for selective access to the interior of an organ, such as the heart in this example, but also prevent egress of fluid from the organ and provide for a secure closure of the access puncture when the procedure is complete.

Such closure may be especially important in certain situations. For example, if a gall bladder is inadvertently perforated during either minimally invasive or traditional surgery, bile escapes and may contaminate other organs and tissues in the abdominal cavity. It is imperative, therefore, that the accidental perforation be closed immediately and completely. In a conventional technique for closing a perforated gall bladder, a clamp is attached to the organ at the area of perforation. A flexible loop (e.g., a suture) is then passed around the clamp and drawn shut. This technique is difficult and time-consuming. Moreover, a significant quantity of bile generally escapes the bladder into the abdomen during the manual manipulation and closure.

Another kind of surgery requiring rapid and thorough control of perforations in internal tissues arises when a patient is a victim of a violent attack or serious accident. In such severe trauma cases, organs often become perforated and release blood into the patients abdominal cavity. The closure of such wounds must be effectuated as quickly and efficiently as possible to minimize internal bleeding, though a surgeon may also require a continued ability to access the interior of the organ once the emergent bleeding issues have been addressed but before the surgical procedure is complete.

### SUMMARY OF THE INVENTION

In an embodiment of the present invention, an apparatus for selectively permitting access through a body tissue is described. The body tissue has oppositely disposed first and second tissue sides with an aperture extending therebetween. A proximal balloon is adapted for placement adjacent the first tissue side. A distal balloon is adapted for placement adjacent the second tissue side. An expandable support member is adapted to extend through the aperture and defines a support member lumen providing fluid communication between the first and second tissue sides. The expandable support member is attached to the proximal and distal balloons and has at least one of an inner support member lining and an outer support member cover. A support member valve is located within the support member lumen and selectively permits access through the aperture between the first and second tissue sides. A closure device is provided for permanently occluding the support member lumen.

In an embodiment of the present invention, an access device for a body tissue is described. The body tissue has oppositely disposed first and second tissue sides with an aperture extending therebetween. Means for defining a lumen through the aperture are provided. Means for padding the means for defining are provided, the means for padding being at least partially located in contact with both the means for defining and the aperture. Means for retaining the means for defining within the aperture are provided, the means for retaining being inflatable. Means for selectively permitting fluid communication through the lumen between the first and second tissue sides are provided. Means for permanently occluding the lumen are provided.

In an embodiment of the present invention, a method of performing a surgical procedure through a body tissue is described. The body tissue has oppositely disposed first and second tissue sides with an aperture extending therebetween. An expandable support member is provided. The expandable support member has a support member lumen with at least one of an outer support member cover and an inner support member lining. The expandable support member is extended through the aperture. Fluid communication is provided between the first and second tissue sides through the support member lumen. A proximal balloon is inflated adjacent the first tissue side. The proximal balloon is attached to a first end of the expandable support member. A distal balloon is inflated adjacent the second tissue side. The distal balloon is attached to a second end of the expandable support member spaced apart from the first end. Fluid communication is selectively permitted through the support member lumen. At least one surgical tool is extended through the support member lumen to conduct the surgical procedure through the aperture. The at least one surgical tool is removed from the support member lumen. The support member lumen is permanently occluded.

### BRIEF DESCRIPTION OF THE DRAWINGS

For a better understanding of the invention, reference may be made to the accompanying drawings, in which:

FIG. 1 is a schematic side view of one embodiment of the present invention;

FIG. 2 is a cross-sectional view taken along line 2-2 of FIG. 1, with the embodiment of FIG. 1 in a first condition;

FIG. 3 is a cross-sectional view similar to that of FIG. 2, with the embodiment of FIG. 1 in a second condition;

FIG. 4A is a cross-sectional view taken along line 4-4 of FIG. 3, with the embodiment of FIG. 1 in a first configuration;

FIG. 4B is a cross-sectional view taken along line 4-4 of FIG. 3, with the embodiment of FIG. 1 in a second configuration;

FIG. 4C is a cross-sectional view taken along line 4-4 of FIG. 3, with the embodiment of FIG. 1 in a third configuration;

FIG. 4D is a cross-sectional view taken along line 4-4 of FIG. 3, with the embodiment of FIG. 1 in a fourth configuration;

FIG. 4E is a cross-sectional view taken along line 4-4 of FIG. 3, with the embodiment of FIG. 1 in a fifth configuration;

FIG. 4F is a cross-sectional view taken along line 4-4 of FIG. 3, with the embodiment of FIG. 1 in a sixth configuration; and

FIGS. 5A-5F illustrate a sequence of operation of the embodiment of FIG. 1.

### DESCRIPTION OF EMBODIMENTS

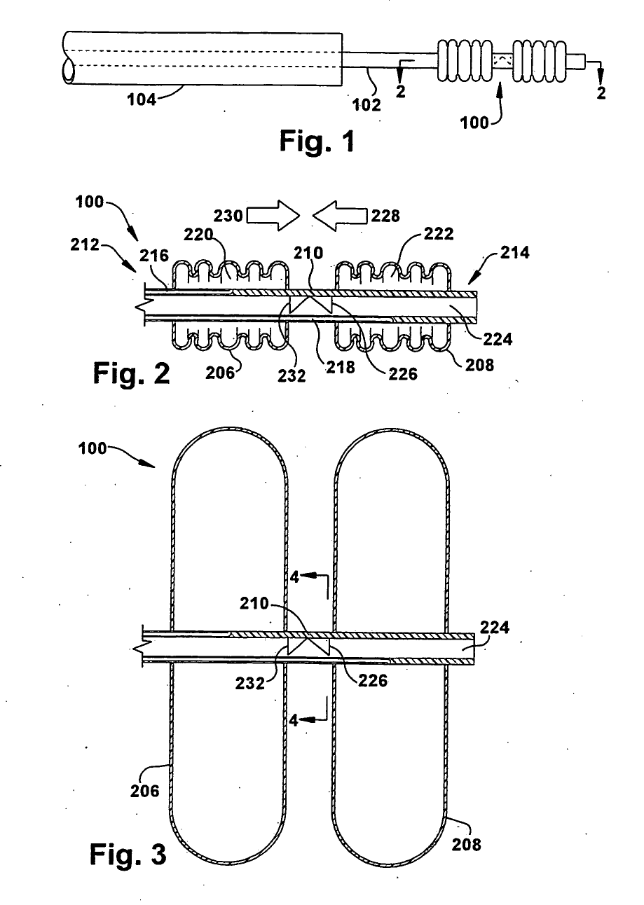

In accordance with the present invention, FIG. 1 depicts an apparatus 100 for selectively permitting access through a body tissue. The apparatus 100 will hereafter be referenced as an access device 100 but may also or instead be used to perform a sealing function without necessarily permitting access through the body tissue.

The access device 100 is shown in FIG. 1 as extending from a delivery catheter 102. The delivery catheter 102 is removably attached to the access device 100, such as through a releasable coupling (not shown) which may be remotely operated to release the access device at a desired time in any suitable manner; FIG. 1 also depicts an insertion tube 104 through which the delivery catheter 102 may be inserted into the body and to the desired access-location for use of the access device 100. Together the insertion tube 104 and delivery catheter 102 comprise an implantation device, to which the access device 100 may be selectively attached, such as through a releasable coupling. The implantation device may be readily provided by one of ordinary skill in the art for a particular application of the present invention, and will not be discussed in further detail herein.

A cross-sectional view of the access device 100, taken along line 2-2 of FIG. 1, is shown in FIG. 2. The access device 100 includes a proximal balloon 206, a distal balloon 208, and an expandable support member 210 having proximal and distal support member ends 212 and 214, respectively. In the view of FIG. 2, the proximal and distal balloons 206 are both depicted in a first, collapsed condition.

The proximal and distal balloons 206 and 208 are attached to the expandable support member 210 adjacent respective ones of the proximal and distal support member ends 212 and 214. It is contemplated that the proximal and distal balloons 206 and 208 may be of any suitable type and need not be of the same type. For example, at least one of the proximal and distal balloons 206 and 208 may include a semi-rigid structure, such as an additional expandable support member (not shown), which need not resemble the expandable support member 210. Proximal and distal balloon inflation lumens 216 and 218, respectively, are associated with the expandable support member 210 and provide fluid communication between one or more outside inflation fluid sources (not shown) and respective proximal and distal inner cavities, 220 and 222, of the proximal and distal balloons 206 and 208. The proximal and distal balloon inflation lumens 216 and 218 may be formed integrally with the expandable support member 210 or may be separately provided. The proximal and distal balloon inflation lumens 216 and 218 may be designed to be separable from the access device 100 if desired.

The proximal and distal balloons 206 and 208 may be inflated and/or deflated in any suitable sequence (simultaneously or separately) and at any desired relative rates. The proximal and distal balloons 206 and 208 may be formed separately or integrally. In the latter case, the proximal and distal inner cavities 220 and 222 may be in fluid communication with one another and therefore may be inflatable in an interdependent manner. Optionally, and regardless of the construction/inflation of the proximal and distal balloons 206 and 208, an inflation coupling or valve/connector system (not shown) may facilitate such fluid communication in a manner that allows the access device 100 to be detached from the implantation device without affecting the inflation state of either of the proximal and distal balloons.

The expandable support member 210, which may be a rigid expandable support member, defines a support member lumen 224 providing fluid communication between the proximal and distal support member ends 212 and 214. A support member valve 226 is located within the support member lumen 224 and selectively permits access through the support member lumen. The support member valve 226 is shown schematically in the Figures as being a solid-walled tube, for ease of depiction, but may be of any suitable type. For example, the expandable support member 210 could be made of plastic, metal, and/or other self-expanding or manually expandable materials, and in any desired configuration such as, but not limited to, a woven or mesh-like structure, optionally having an open/perforated structure.

The support member valve 226 may be of any suitable type. For example, the support member valve 226 may be mechanically operable as an iris diaphragm (that is, like the aperture of an optical lens). Alternatively, the support member valve 226 may be constructed of a self-healing diaphragm or an elastic material with a small central opening that can be dilated by objects inserted therethrough, but which maintains a substantially fluidtight seal with the inserted object and substantially closes when the object is withdrawn.

One or more support member valves 226 could be associated with the support member lumen 224; multiple support member valves in series may assist in providing desired fluid communication characteristics. Similarly, multiple support member valves 226 in parallel may assist in preventing unwanted fluid communication as multiple surgical tools are inserted into, and withdrawn from, the support member lumen 224. For simplicity in description, however, a single “support member valve” referenced herein will be considered to encompass any such multiple valve arrangements as desired for a particular application of the present invention.

Optionally, the support member valve 226 is a unidirectional support member valve, adapted to permit passage of certain substances in a first direction (arrow 228 in FIG. 2) and to prevent passage of certain substances in a second direction (arrow 230 in FIG. 2) opposite the first direction. The permitted/prevented substances need not be the same; for instance, the support member valve 226 could be adapted to allow advancement of surgical tools in the first direction 228 and withdrawal of the surgical tools in the second direction 230 while substantially preventing fluid from passing through the support member lumen 224 in the second direction. Likewise, the support member valve 226 could be adapted to allow a surgical tool, for example, to be inserted in the first direction 228 and extend therethrough for an extended period of time (i.e., the length of time required for an internal surgical procedure carried out using the access device 100) while concurrently preventing blood from passing through the support member lumen 224 in the second direction 230.

The expandable support member 210 may include a closure device 232 adapted for selectively occluding the support member lumen 224. The closure device 232, shown schematically in the Figures, may be of any suitable type and may be formed integrally with the expandable support member 210 or may be provided separately, at any time before, during, or after use of the expandable support member. Optionally, the closure device 232 may be of a reversible type, but it is contemplated that the closure device 232 may be operative to permanently occlude the support member lumen 224 in most applications of the present invention. For example, the closure device 232 may be a locking structure, a mass of an obstructive material, or have any other suitable configuration. The closure device 232 may also or instead be provided without a dedicated or separate locking structure by simply placing the support member valve 226 in an occlusion arrangement/position.

FIG. 3 depicts the access device 100 with each of the proximal and distal balloons 206 and 208 inflated to a second, expanded condition, in contrast to the collapsed condition of the proximal and distal balloons shown in FIG. 1. The proximal and distal balloons 206 and 208 may be inflated using any suitable gas or liquid, which may be selectively supplied by a user through the proximal and distal balloon inflation lumens 216 and 218, respectively. At least one of the proximal and distal balloons 206 and 208 may be adapted for substantially permanent affixation in the expanded condition, as will be discussed below in more detail.

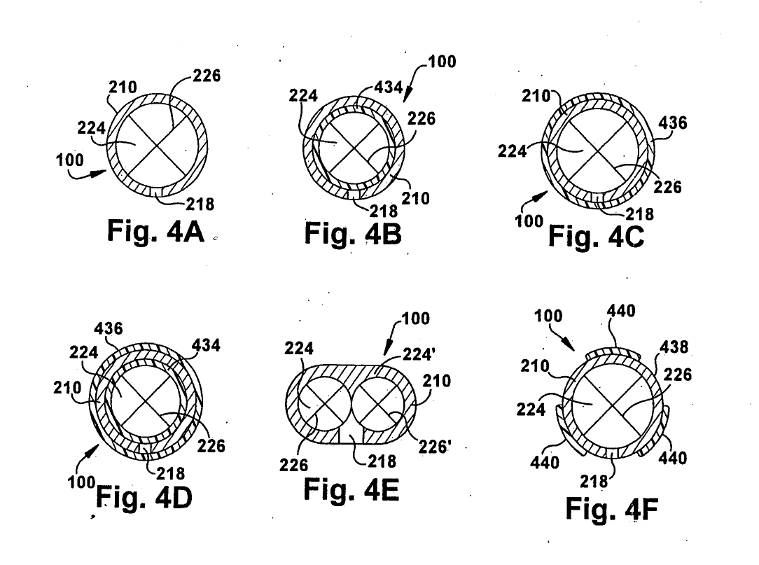

The expandable support member 210 could have any of several configurations, with various adjacent structures being optionally present, as desired for a particular application of the present invention. Examples of some suitable configurations are shown in FIGS. 4A-4F. FIG. 4A depicts a cross-sectional view taken along line 4-4 in FIG. 3, with the depicted structures in a first configuration. FIGS. 4B-4F show similarly placed cross-sections of alternate (i.e., second through sixth) configurations of the access device 100, which will each be discussed in turn.

Returning to FIG. 4A, the access device 100 is shown in a simple form, in which the expandable support member 210 surrounds the support member valve 226 and defines the support member lumen 224 with no intervening members.

Turning to FIG. 4B, a second optional configuration of a suitable structure for the access device 100 is shown. In FIG. 4B, the expandable support member 210 is lined with an inner support member lining 434.

In FIG. 4C, a third optional configuration of a suitable structure for the access device 100 is shown. In FIG. 4C, the expandable support member 210 is covered with an outer support member cover 436.

In FIG. 4D, a fourth optional configuration of a suitable structure for the access device 100 is shown. In FIG. 4D, the expandable support member 210 is both lined with an inner support member lining 434 and covered with an outer support member cover 436.

In FIG. 4E, a fifth optional configuration of a suitable structure for the access device 100 is shown. In FIG. 4E, the expandable support member 210 defines both a support member lumen 224 and an alternate support member lumen 224′. The alternate support member lumen 224′ may include an associated alternate support member valve 226′, as shown. Though two lumens are shown in FIG. 4E, any desired number of lumens could be defined by the expandable support member 210 and may readily be provided by one of ordinary skill in the art.

In FIG. 4F, a sixth optional configuration of a suitable structure for the access device 100 is shown. In FIG. 4F, the expandable support member 210 has an outer support member wall 438, which includes at least one padded portion 440. The padded portion 440 could be at least partially formed integrally with the outer support member wall 438, or could be separately provided. The padded portion 440 could be located adjacent any suitable portion of the outer support member wall 438 and, as shown in FIG. 4F, need not extend continuously in any direction along the expandable support member 210.

The access device 100 could include any desired combination of multiple support member lumens 224 and 224′, an inner support member lining 434, an outer support member cover 436, and/or at least one padded portion 440. The configurations shown in FIGS. 4A-4F are intended for illustrative purposes only and are neither intended nor operative to limit the present invention. The inner support member lining 434, outer support member cover 436, and/or padded portion(s) 440 may be at least partially formed from any suitable material such as, but not limited to, peritoneal tissue, pericardial tissue, pleural tissue, fascia tissue, and artificial issue. When present, the inner support member lining 434, outer support member cover 436, and/or padded portion(s) may be formed integrally with the expandable support member 210 or provided to the expandable support member at any time before, during, or after use of the access device 100.

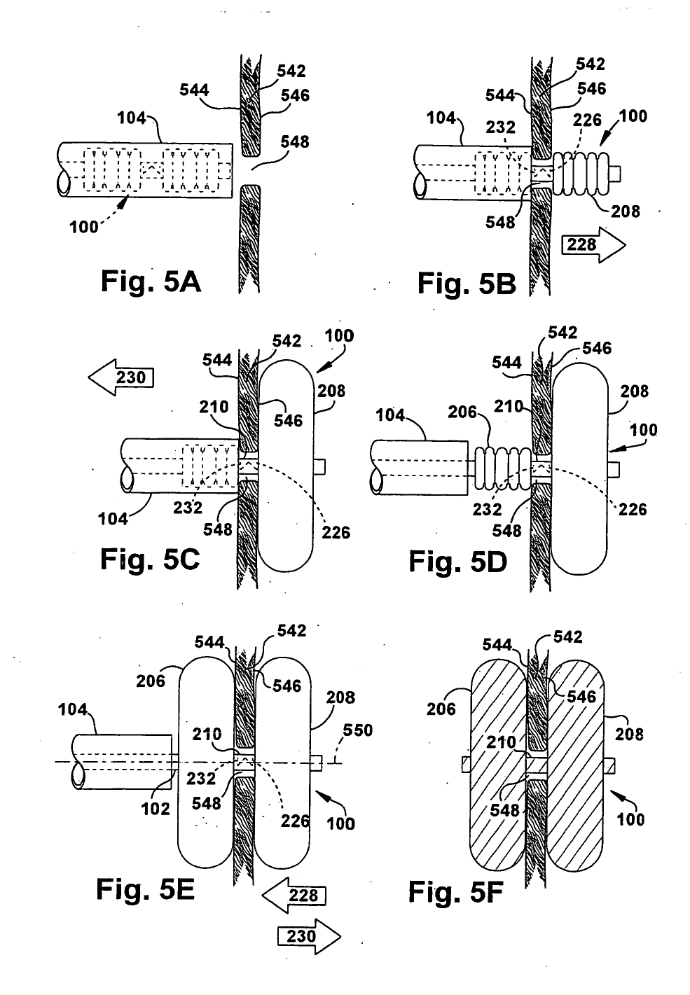

FIGS. 5A-5F illustrate a sequence of operation of the access device 100, which selectively permits access through a body tissue 542. The access device 100 is shown schematically herein for ease of depiction, and may include structures and features, such as the proximal and distal balloon inflation lumens 216 and 218, which are not shown in FIGS. 5A-5F. The body issue 542 may be at least one of a cardiac tissue, a peritoneal tissue, a pleural tissue, a skin tissue, another organ wall tissue, or any other body tissue. The body tissue 542 has oppositely disposed first and second tissue sides 544 and 546, respectively, with an aperture 548 extending therebetween. The aperture 548 may be naturally or artificially formed in the body tissue 542, and may be created at any time. For example, a puncturing member (not shown) could be passed through the insertion tube 104 and pierce the body tissue 542 to create the aperture 548 immediately before deployment of the access device 100.

As shown in FIG. 5B, the access device 100 may be placed into engagement with the body tissue 542 by extension of the expandable support member 210 through the aperture 548 in the first direction 228. The expandable support member 210 provides fluid communication between the first and second tissue sides 544 and 546 through the support member lumen 224. Selective fluid access through the aperture 548 between the first and second tissue sides 544 and 546 is provided and/or controlled by the support member valve 226. Optionally, the expandable support member 210, the proximal balloon 206, the distal balloon 208, and/or any included inner support member lining 434 or outer support member cover 436 may be chosen, sized, and/or designed to substantially restrict fluid flow through the aperture 548 to a flow path through the support member lumen 224. In other words, various structures of the access device 106 may be dimensioned to fit snugly with the body tissue 542 and substantially occlude fluid paths through the aperture 548 other than via the support member lumen 224.

The proximal balloon 206 is adapted for placement adjacent the first tissue side 544 and the distal balloon 208 is adapted for placement adjacent the second tissue side 546. Optionally, the expandable support member 210 may be placed within the aperture 548 with the distal balloon 208 in the collapsed condition shown in FIG. 5B, for ease of insertion.

As shown in FIG. 5C, the distal balloon 208 may be inflated into the expanded condition adjacent the second tissue side 546. As an example, the distal balloon 208 is shown herein as being inflated while a portion of the access device 100 is still within the insertion tube 104, which may provide some “anchoring” properties. However, the proximal and distal balloons 208 can each be inflated, partially or fully, at any suitable time and in any suitable sequence as desired by the user and with no restriction imposed by the relative positioning of the access device 100, the insertion tube 104 or other delivery device, and/or the aperture 548 or body tissue 542; the sequence shown in FIGS. 5A-5F is given for example purposes only.

Presuming that the distal balloon 208 is inflated in FIG. 5C, the insertion tube 104 may be moved in the second direction 230 to expose the proximal balloon 206, as shown in the collapsed condition in FIG. 5D. The proximal balloon 206 may be inflated into the expanded condition, as shown in FIG. 5E. Optionally, at least one of the proximal and distal balloons 206 and 208 may exert a compressive force upon the body tissue 542. For example, the proximal and distal balloons 206 and 208 may slightly squeeze the body tissue 542 therebetween when in the expanded condition and thereby attach the access device 100 firmly to the body tissue.

In FIG. 5E, the delivery catheter 102 remains attached to the access device 100, and a guidewire 550 is depicted as representative of structures/substances which may be passed through the support member lumen 224 in the first direction 228. Other examples of objects which may be inserted through the access device 100 include catheters, endoscopes, needles, other surgical tools, or the like, or any combinations thereof. Additionally or alternatively, fluids such as oxygen, saline, pharmaceutical agents, or any other fluids could pass through the support member lumen 224 in the first direction 228. The support member valve 226 may operate in a unidirectional manner to allow fluid communication (and passage of objects/substances) through the expandable support member 210 in the first direction 228 while preventing fluid communication (e.g., blood) through the expandable support member 210 in the second direction 230. Optionally, though, even a unidirectional support member valve 226 could be configured to allow selectively controlled fluid communication in the second direction 230, such as through a vacuum catheter (not shown) or if fluid pressure adjacent the second tissue side 546 exceeds a predetermined value (to thereby provide a shunting function for undesirably high pressures).

When the access device 100 is configured and arranged as shown in FIG. 5E, a surgical procedure can be performed through the body tissue 542. For example, when the body tissue 542 is a heart wall, the access device 100 could be placed within an apical puncture aperture 548 and therefore facilitate a percutaneous heart valve repair or another surgical procedure within the heart. One of ordinary skill in the art could readily perform a desired surgical procedure through the body tissue 542 using the access device 100, deployed as shown in FIG. 5E, and any combination of percutaneous and/or conventional surgical tools and devices. It is contemplated that the user may detach the access device 100 from the delivery catheter 102 during use, possibly in cooperation with a guidewire 550 which remains within the support member lumen 224 to aid the provision of an alternate catheter (not shown) to the access device 100 using an exchange technique.

When the user no longer desires access through the aperture 548 (i.e., at the conclusion of the surgical procedure being conducted therethrough), the closure device 232 may be actuated to occlude the support member lumen 224, which is shown schematically in FIG. 5F with cross-hatching to indicate that the closure device is operative to close fluid communication through the aperture. FIG. 5F also depicts the access device 100 in a freestanding state, after having been detached from the implantation device.

In FIG. 5F, both of the proximal and distal balloons 206 and 208 are shown, again via cross-hatching, as having been permanently affixed in the expanded condition. Neither, either one, or both of the proximal and distal balloons 206 and 208 may be adapted for permanent affixation in an at least partially expanded condition, as desired for a particular application of the present invention. Such permanent affixation may be provided in any suitable manner, and using any desired affixation treatment(s)/method(s) and/or supplemental agent(s). The chosen mechanism(s) may operate upon the proximal and/or distal balloons 206 and 208 themselves or upon/via one or more supplemental agents, such as a material located or placed within the proximal and/or distal inner cavities 220 and 222. Supplemental agents, when present, may be originally present when the access device 100 is deployed or may be provided to the proximal and/or distal balloons 206 and 208 at any time before the permanent affixation is effected. For example, the proximal and/or distal balloon inflation lumens 216 and 218 may be used to provide a supplemental agent to the respective balloon, either originally as an inflation fluid during deployment of the access device 100 or at a later time when permanent affixation is desired.

Permanent affixation, when present, may be at least partially provided to at least one of the proximal and distal balloons 206 and 208 through at least one of the following mechanisms: a change in pH concentration, a change in ionic concentration, a change in state of an adhesive or gel, an organic solvent, permeation of a secondary material capable of precipitation, exposure to heat energy, exposure to light energy, exposure to laser energy, exposure to cooling energy, exposure to a gas, exposure to a liquid, exposure to pressure, the passage of time, and a mechanical retention structure. When both of the proximal and distal balloons 206 and 208 are adapted for permanent affixation in the expanded condition, the same method need not be used to affix both. Some examples of suitable methods and supplemental agents which may be used to permanently affix one or both of the proximal and distal balloons 206 and 208 in the expanded condition are given in U.S. Pat. No. 5,634,936, issued Jun. 3, 1997 to Bradley Linden et al.

It is contemplated that at least a portion of the access device 100 may be formed from a material operative to encourage biological ingrowth of the body tissue 542 to the material. For example, at least a portion of the proximal and/or distal balloons 206 and 208 could be made of a textured material or coated with an agent to encourage endothelialization. Additionally or alternatively, at least the access device 100 could include an adhesive portion adapted to adhere at least a portion of the access device to the body tissue 542. The padded portion 440 could be formed by a thickened wall portion of the expandable support member 210, could result from the attachment of a padding structure (not shown) to the expandable support member 210, or could be formed in any suitable manner, integrally with or separately from the expandable support member. This adhesive portion could be any of the described structures enhanced with an adhesive coating or could be a separately provided structure (not shown)

It is also contemplated that the access device 100 or any portion thereof may be adapted to elute a therapeutic agent, such as, but not limited to, an anesthetic, anti-inflammatory, or antiseptic agent, to the body tissue 542. “Elute” is used herein to indicate that a therapeutic agent is released, leached, diffused, or otherwise provided to the target tissue.

Herein, “permanent” means “continuing or enduring without fundamental or marked change; stable”; however, any condition described as “permanent” could be reversed by a user at a later time through the action of an external agent. For example, an obstructing mass within the support member lumen 224 could be reamed out in a subsequent reoperation, or a hardened-polymer within an expanded proximal or distal balloon 206 or 208 could be dissolved to allow the balloon to be returned to the deflated condition.

While aspects of the present invention have been particularly shown and described with reference to the preferred embodiment above, it will be understood by those of ordinary skill in the art that various additional embodiments may be contemplated without departing from the spirit and scope of the present invention. For example, the support member valve 226 may include no, or any number of, moving parts (e.g., the support member valve could be a simple self-healing membrane). The proximal and/or distal balloons 206 and 208 may be held for any desired length of time in any of a plurality of intermediate inflation positions between fully collapsed and fully expanded states. The structures forming the access device 100 could be made of any suitable material or combination of materials, though it is preferred for the access device 100 to be at least partially formed from biocompatible materials. The insertion tube 104 or another delivery structure (not shown) may create, and/or extend through, the aperture 548 before or during deployment of the access device 548 into the body tissue 548. One or both of the proximal and distal balloons 206 and 208 could be supplied and deployed from at least partially within the expandable support member 210. The access device 100 is described as being used in a cardiac application, but could also or instead be used in any other suitable surgical procedure. At the conclusion of the surgical procedure, the access device 100 need not remain within the patient's body but may be removed, for instance, by reversing the steps shown in FIGS. 5A-5D; the aperture 548 could then be closed in any suitable manner. A proximal or distal balloon 206 or 208 being permanently affixed in the expanded condition may be at least partially deflated, inflated, and/or reinflated before and/or during the affixation process, optionally multiple times. A single one of the inner support member lining 434, outer support member cover 436, and/or padded portion 440 may perform one or more functions of the other ones. At least a portion of the access device 100 may be biodegradable and adapted to degrade after a predetermined time period. An inflation fluid and/or supplemental agent could be provided to one or more of the proximal and distal balloons 206 and 208 through means other than the proximal and distal balloon inflation lumens 216 and 218, respectively. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

Having described the invention, we claim:

1. An apparatus for selectively permitting access through a body tissue, the body tissue having oppositely disposed first and second tissue sides with an aperture extending therebetween, the apparatus comprising:
a proximal balloon adapted for placement adjacent the first tissue side;
a distal balloon adapted for placement adjacent the second tissue side;
an expandable support member adapted to extend through the aperture and defining a support member lumen providing fluid communication between the first and second tissue sides, the expandable support member being attached to the proximal and distal balloons and having at least one of an inner support member lining and an outer support member cover;
a support member valve located within the support member lumen and selectively permitting access through the aperture between the first and second tissue sides; and
a closure device for permanently occluding the support member lumen.

2. The apparatus of claim 1, wherein each of the proximal and distal balloons is selectively inflatable between a first, collapsed condition and a second, expanded condition, and the expandable support member is placed within the aperture with the distal balloon in the collapsed condition.

3. The apparatus of claim 2, wherein at least one of the proximal and distal balloons is adapted for permanent affixation in the expanded condition.

4. The apparatus of claim 3, wherein the permanent affixation is at least partially provided to the at least one of the proximal and distal balloons through at least one of: a change in pH concentration, a change in ionic concentration, an organic solvent, permeation of a secondary material capable of precipitation, exposure to heat energy, exposure to light energy, exposure to laser energy, exposure to cooling energy, exposure to a gas, exposure to a liquid, exposure to pressure, the passage of time, and a mechanical retention structure.

5. The apparatus of claim 1, wherein the support member valve is a unidirectional support member valve.

6. The apparatus of claim 1, wherein the proximal and distal balloons are formed integrally.

7. The apparatus of claim 1, wherein at least one of the proximal and distal balloons exerts a compressive force upon the body tissue.

8. The apparatus of claim 1, wherein at least one of the proximal and distal balloons includes a semi-rigid structure.

9. The apparatus of claim 1, wherein the proximal balloon has a proximal inner cavity, the distal balloon has a distal inner cavity, and the proximal and distal inner cavities are in fluid communication.

10. The apparatus of claim 1, wherein the expandable support member defines at least two support member lumens.

11. The apparatus of claim 1, including a releasable coupling to selectively attach the apparatus to an implantation device.

12. The apparatus of claim 1, wherein at least a portion of the apparatus is formed from a material operative to encourage biological ingrowth of the body tissue to the material.

13. The apparatus of claim 1, wherein at least a portion of the apparatus is adapted to elute a therapeutic agent.

14. The apparatus of claim 1, including an adhesive portion adapted to adhere at least a portion of the apparatus to the body tissue.

15. The apparatus of claim 1, wherein the body tissue is at least one of a cardiac tissue, a peritoneal tissue, a pleural tissue, and a skin tissue.

16. The apparatus of claim 1, wherein the aperture is artificially formed in the body tissue.

17. The apparatus of claim 1, wherein at least one of the inner support member lining and the outer support member cover is at least partially formed of a tissue selected from the group comprising: peritoneal tissue, pericardial tissue, pleural tissue, fascia tissue, and artificial tissue.

18. The apparatus of claim 1, wherein the expandable support member includes an outer support member wall, and the outer support member wall includes a padded portion adapted to contact at least a portion of the aperture.

19. The apparatus of claim 1, wherein the expandable support member is a rigid expandable support member.

20. An access device for a body tissue, the body tissue having oppositely disposed first and second tissue sides with an aperture extending therebetween, the access device comprising:
means for defining a lumen through the aperture;
means for padding the means for defining, the means for padding being at least partially located in contact with both the means for defining and the aperture;
means for retaining the means for defining within the aperture, the means for retaining being inflatable;
means for selectively permitting fluid communication through the lumen between the first and second tissue sides; and
means for permanently occluding the lumen.

21. The access device of claim 20, wherein the means for retaining is selectively inflatable between a first, collapsed condition and a second, expanded condition, the access device including means for inflating the means for retaining into the expanded condition.

22. The access device of claim 20, including means for eluting a therapeutic agent.

23. The access device of claim 20, wherein the means for selectively permitting fluid communication is means for selectively permitting unidirectional fluid communication through the lumen from the first tissue side toward the second tissue side.

24. A method of performing a surgical procedure through a body tissue, the body tissue having oppositely disposed first and second tissue sides with an aperture extending therebetween, the method comprising the steps of:
providing an expandable support member, having a support member lumen, with at least one of an outer support member cover and an inner support member lining;
extending the expandable support member through the aperture;
providing fluid communication between the first and second tissue sides through the support member lumen;
inflating a proximal balloon adjacent the first tissue side, the proximal balloon being attached to a first end of the expandable support member;
inflating a distal balloon adjacent the second tissue side, the distal balloon being attached to a second end of the expandable support member spaced apart from the first end;
selectively permitting fluid communication through the support member lumen;
extending at least one surgical tool through the support member lumen to conduct the surgical procedure through the aperture;
removing the at least one surgical tool from the support member lumen; and
permanently occluding the support member lumen.

25. The method of claim 24, including the step of contacting at least a portion of the aperture with a padded portion of the expandable support member.

26. The method of claim 24, including the step of preventing fluid flow through the lumen from the second tissue side to the first tissue side.

27. The method of claim 24, including the step of permanently retaining at least one of the proximal and distal balloons in an inflated condition.
